(19) United States
(12) Patent Application Publication
(10) Pub. No.: US 2023/0295156 A1
(43) Pub. Date: Sep. 21, 2023

(54) CRYSTALLINE FORMS OF 4-[(7-CHLORO-2-METHOXYBENZO[B][1,5]NAPHTHYRIDIN-10-YL)AMINO]-2,6-BIS(PYRROLIDIN-1-YLMETHYL)PHENOL AND SALTS THEREOF

(71) Applicant: Merck Patent GmbH, Darmstadt (DE)

(72) Inventors: Axel BECKER, Darmstadt (DE); Delia-Maria Gruia, Darmstadt (DE)

(73) Assignee: Merck Patent GmbH, Darmstadt (DE)

(21) Appl. No.: 17/995,388

(22) PCT Filed: Apr. 14, 2021
(86) PCT No.: PCT/EP2021/059604

§ 371 Date: Oct. 3, 2022

(30) Foreign Application Priority Data
Apr. 17, 2020 (EP) 20170197.6

(51) Int. Cl. C07D 471/04; A61K 45/06

(52) U.S. Cl. CPC C07D 471/04; A61K 45/06; C07B 2200/13

(57) ABSTRACT

Novel crystalline forms of 4-1(7-chloro-2-methoxybenzo[bll 1,5]naphthyridin-10-yl)aminol-2,6-bis(pyrrolidin-l-%Ilmethyl)phenol and salts thereof are useful, in particular for pharmaceutical formulations. Processes can be used for manufacturing the crystalline forms, and the crystalline forms can be used in methods of treatment.

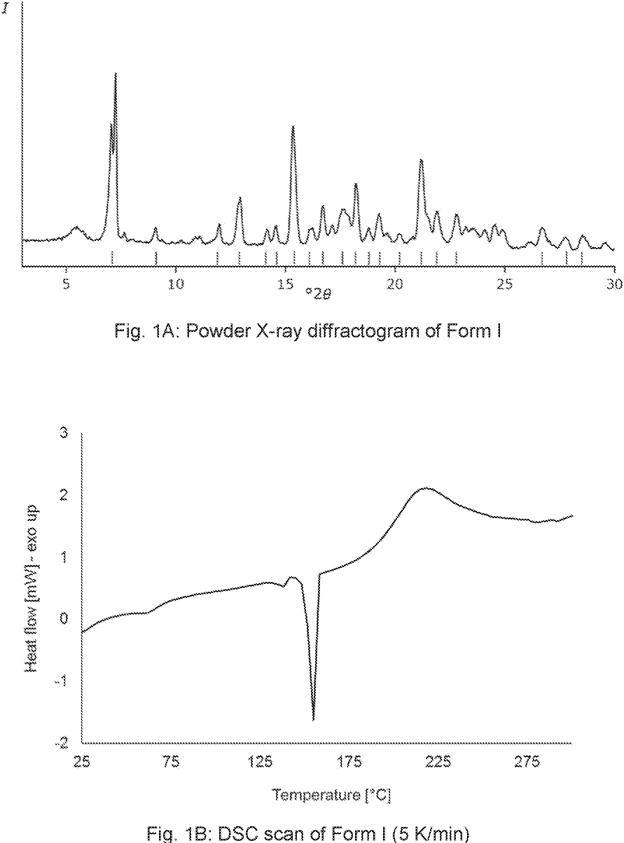

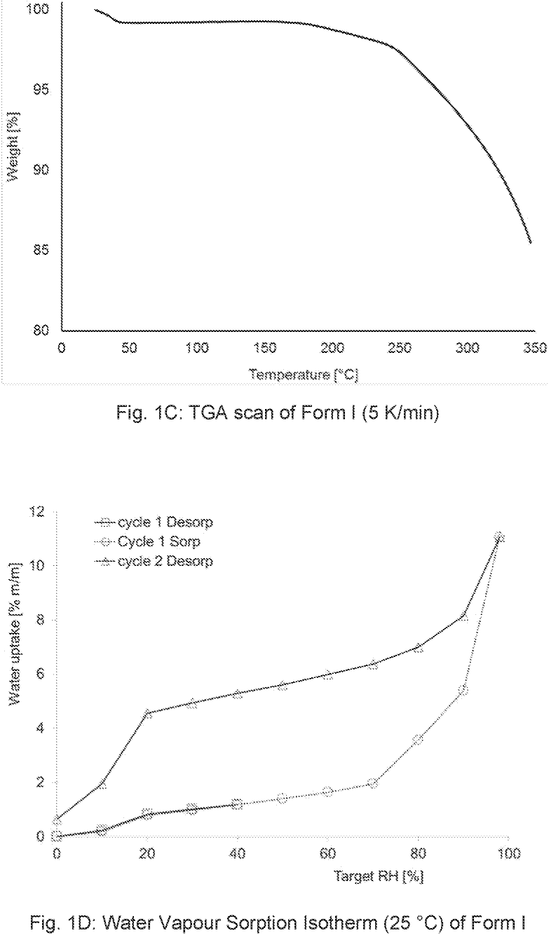

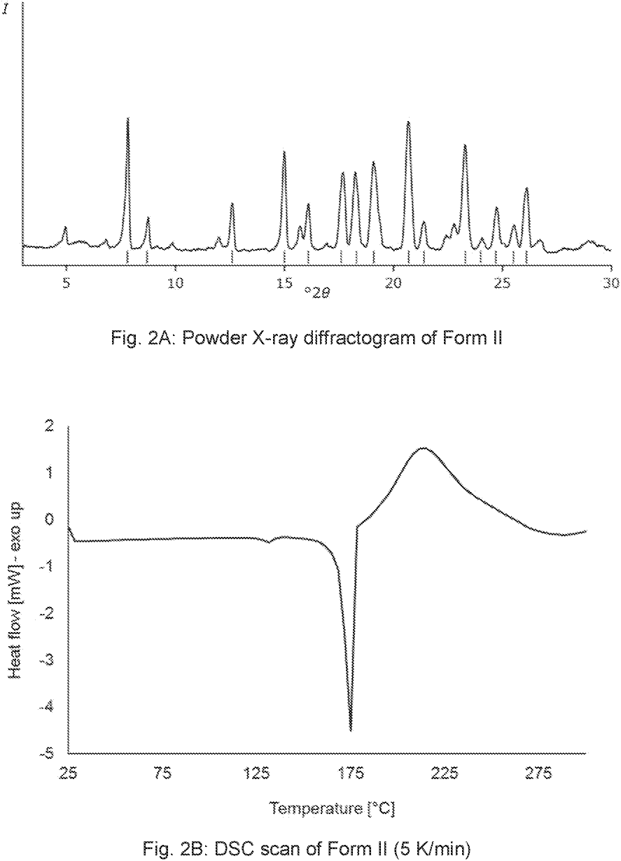

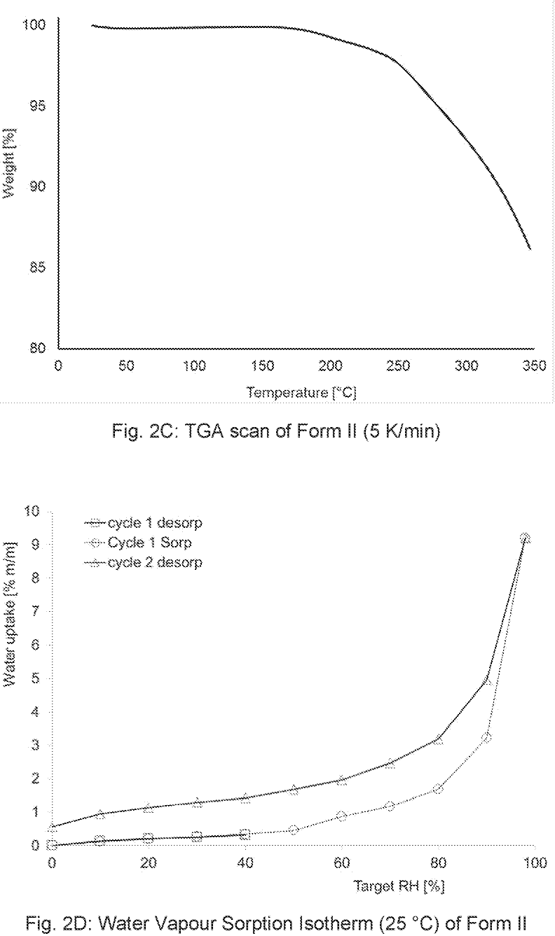

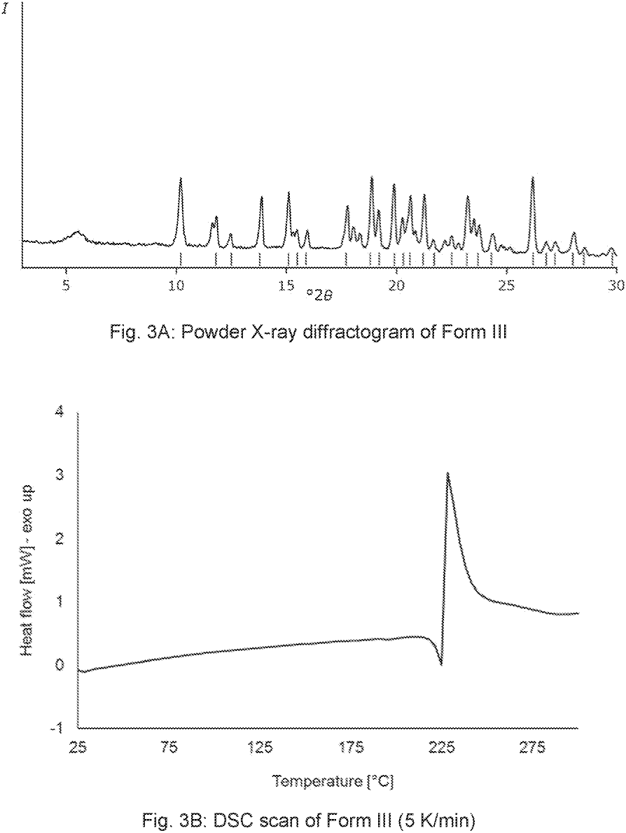

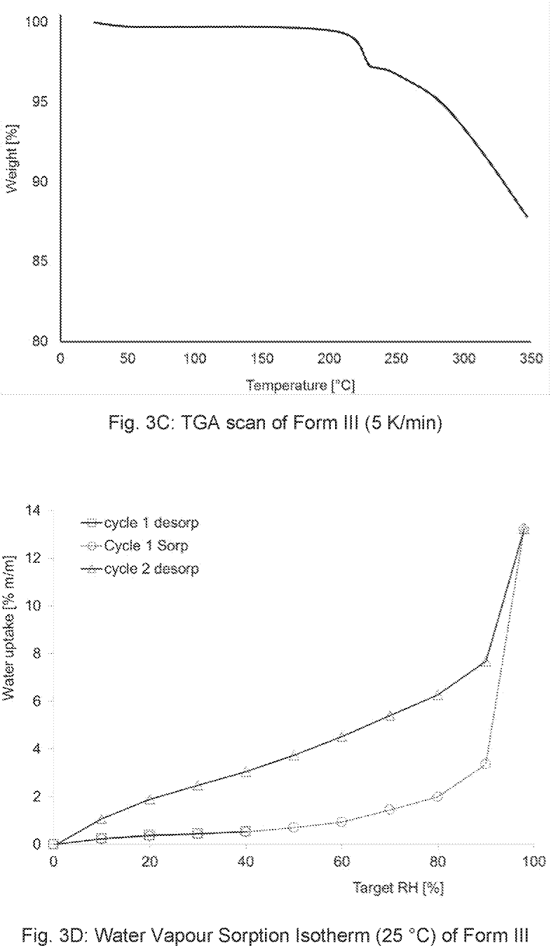

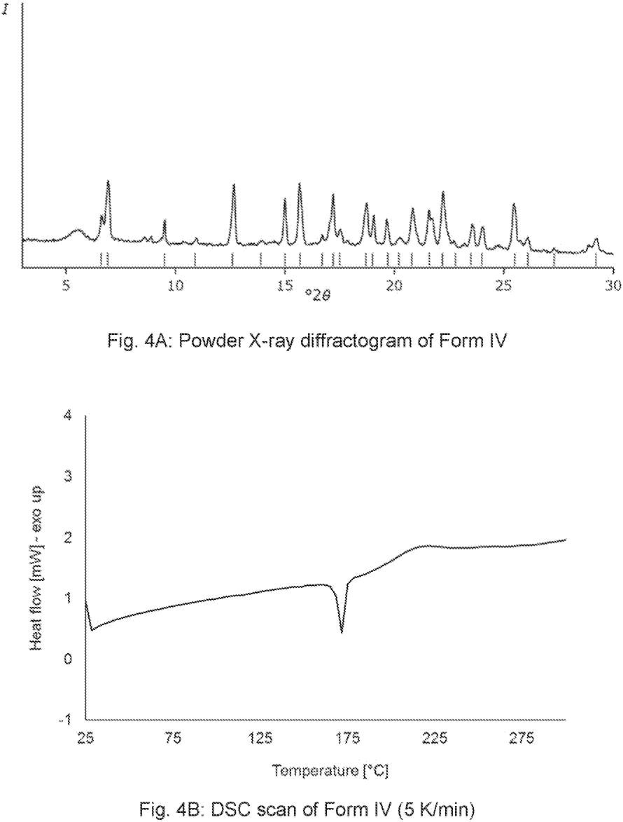

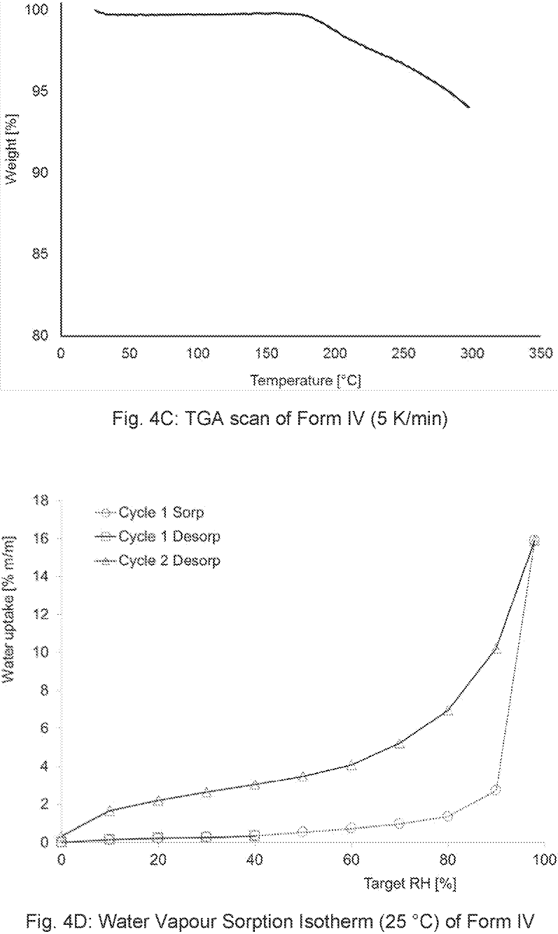

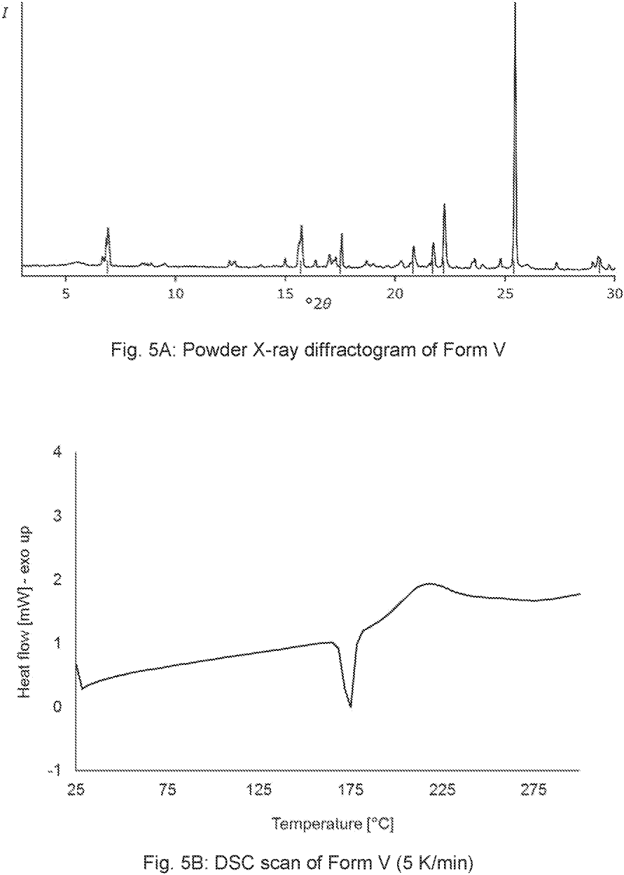

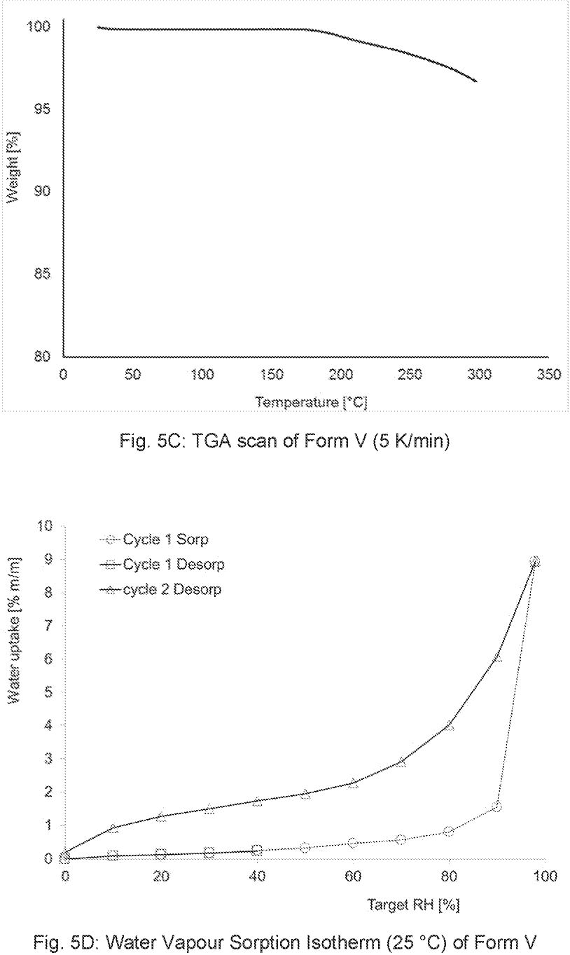

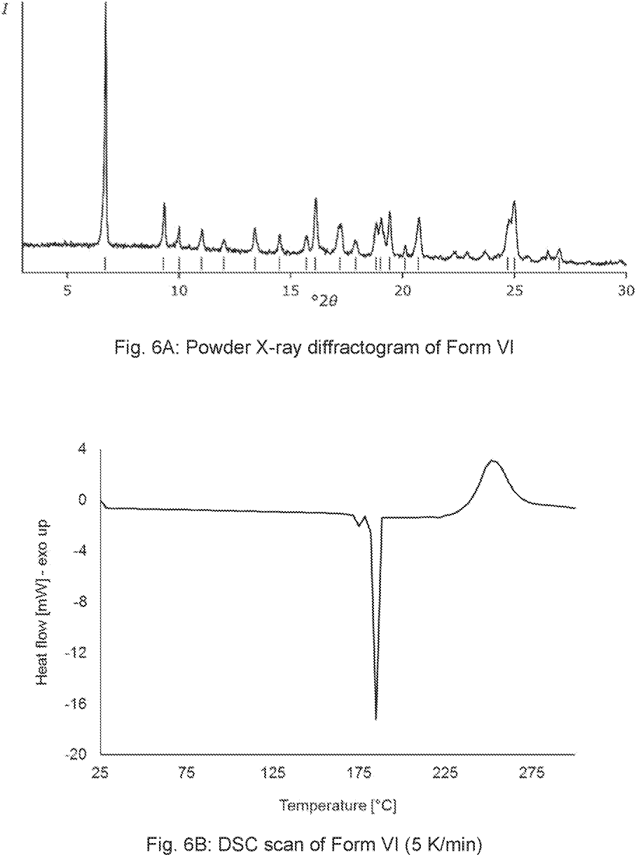

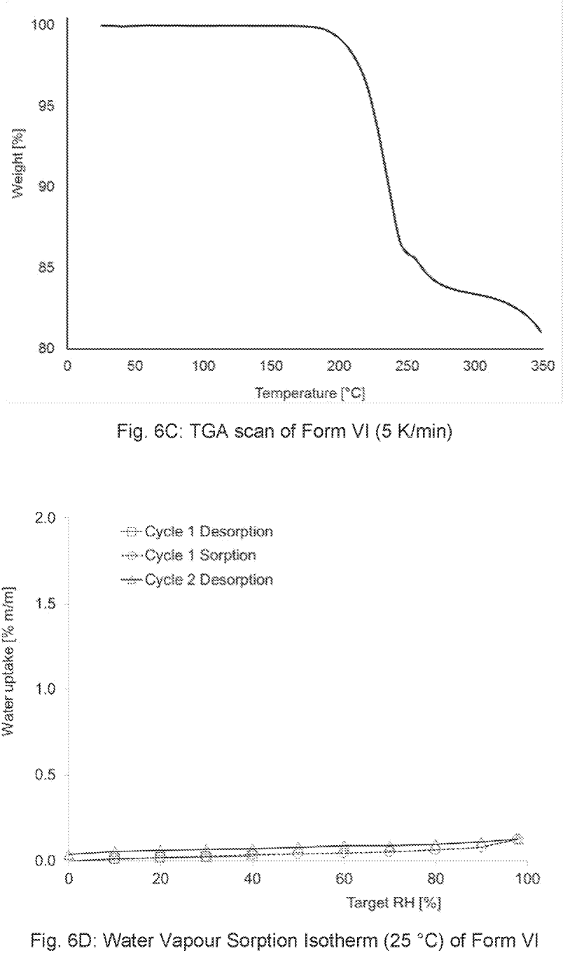

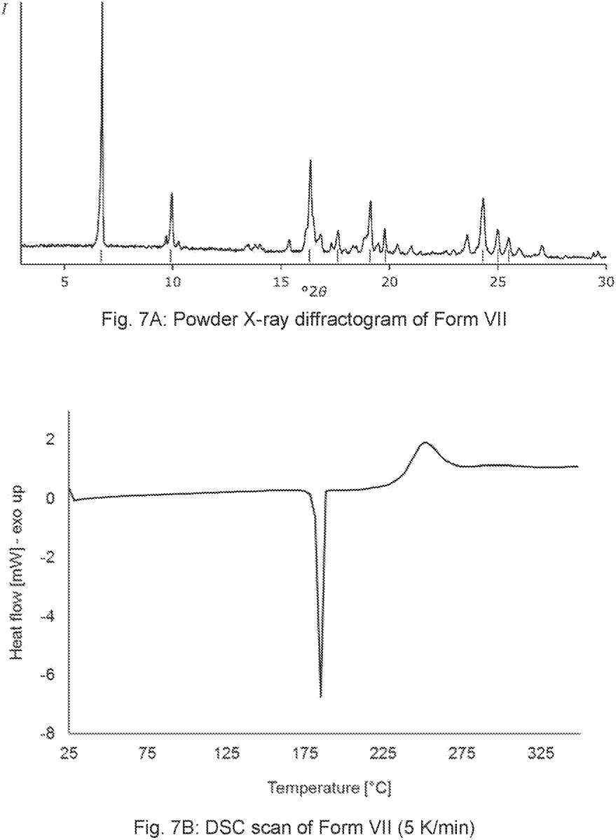

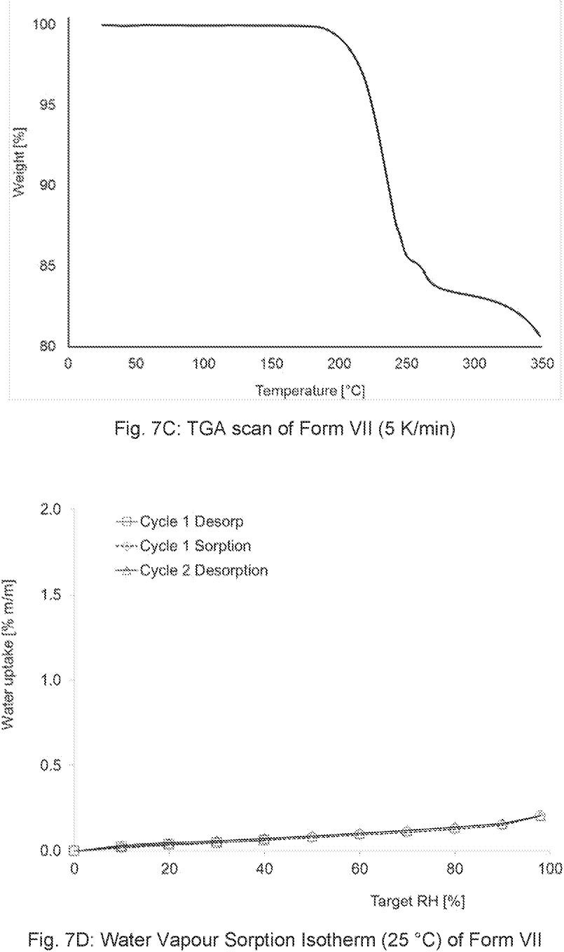

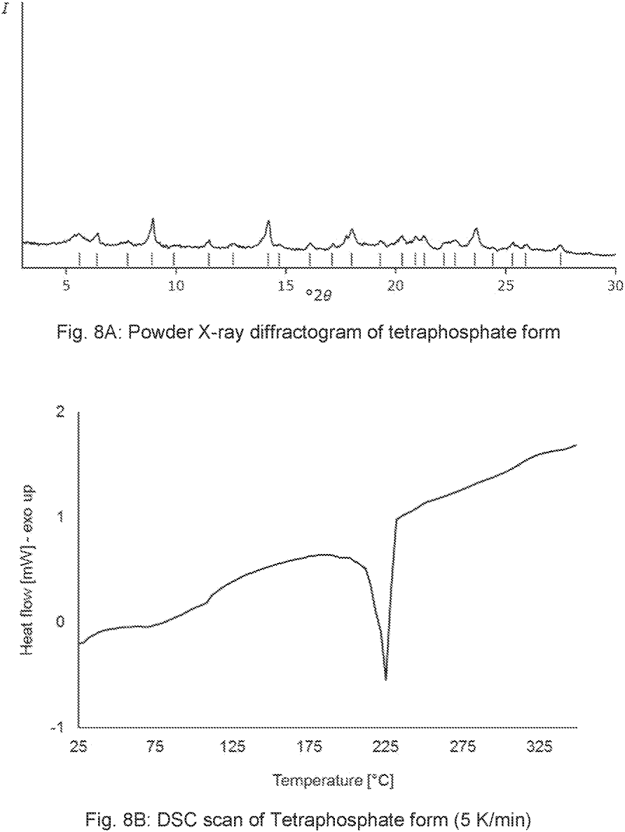

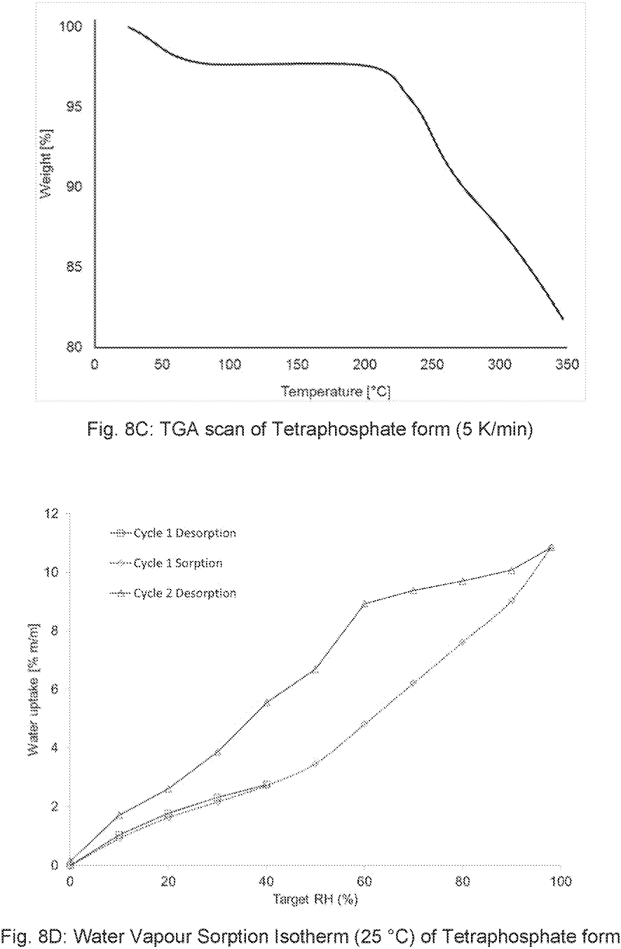

### TECHNICAL FIELD

The present invention relates to novel crystalline of 4-[(7-chloro-2-methoxybenzo[b][1,5]naphthyridin-10-yl)amino]-2,6-bis(pyrrolidin-1 -ylmethyl)phenol and salts thereof, as well as processes of manufacturing the same, and pharmaceutical formulations and uses thereof.

### BACKGROUND OF THE INVENTION

Pyronaridine ((4-[(7-chloro-2-methoxybenzo[b][1,5]naphthyridin-10-yl)amino]-2,6-bis(pyrrolidin-1-ylmethyl)phenol tetraphosphate) is a commercially available antimalarial agent, which was first synthesized in 1970 at the Institute of Chinese Parasitic Disease, Chinese Academy of Preventative Medicine. The commercially available API pyronaridine is the tetraphosphate salt shown below:

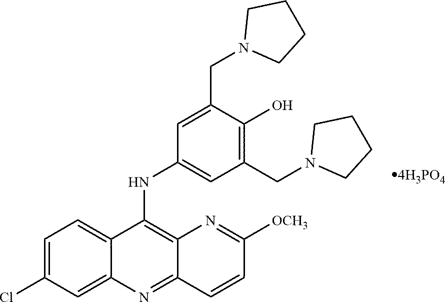

pyronaridine (4-[(7-chloro-2-methoxybenzo[b][1,5]naphthyridin-10-yl)amino]-2,6-bis(pyrrolidin-1-ylmethyl)phenol tetraphosphate)

A tetraphosphate salt monohydrate form of pyronaridine is also described in CN 105461713 A.

For potential use of pyronaridine in fixed-dose combination drug medications (single administration solid-dosage drug product comprising two API drugs), use of the commercially available API (tetraphosphate salt) has certain disadvantages due to high weight fraction of inactive mass in the tetraphosphate salt entity (43 wt% attributed to tetraphosphate counter ion in an anhydrous tetraphosphate salt) and the strong water uptake behavior. The commercially available API is classified as hygroscopic acc. to Ph. Eur. based on water uptake difference between 40% rh and 80% rh (see experimental data described in Examlple 1 of the Experimental Section). The hygroscopicity of pyronaridine is particular disadvantageous in terms of manufacturing behaviour and physical stability behaviour at elevated rel. humidity due to the risk of strongly increased water uptake during drug product manufacturing and storage.

It is known that different salt forms of a compound may have different properties and because of this, different salt forms of a pharmaceutically active ingredient may provide a basis for improving formulation, dissolution profile, stability or shelf-life. Different salts may also give rise to different polymorphic forms, which may provide additional opportunities to improve the properties and characteristics of a pharmaceutical ingredient.

In material science polymorphism is the ability of a solid material to exist in two or more crystal forms that have different arrangements and/or conformations of the molecules in the crystal lattice. Solvates are crystalline solid adducts containing either stoichiometric or non-stoichiometric amounts of a solvent incorporated within the crystal structure. If the incorporated solvent is water, the solvates are also commonly known as hydrates. Polymorphs can be distinguished from one another by different techniques such as powder X-ray diffraction (XRD), thermogravimetric analysis and differential scanning calorimetry. One or more of these techniques may be used to characterize a particular polymorph and to distinguish different polymorphic forms of a compound.

Different polymorphs of a solid material (including solvated forms) can have different properties such as melting point, chemical reactivity, apparent solubility, dissolution rate, vapor pressure, hygroscopicity, particle shape, flowability, compactibility and density. These properties can have a direct effect on the ability to process and manufacture a solid drug substance, as well as on drug product stability, dissolution, and bioavailability. Thus, polymorphism can affect the quality, safety, and efficacy of the drug product. For example, a metastable pharmaceutical solid form can change its crystalline structure or solvate/desolvate in response to changes in environmental conditions or over time. Consequently, stability and shelf life may vary between different polymorphs of a solid substance. New polymorphic forms and solvates of a pharmaceutically useful compound can provide opportunities to improve the performance characteristics of a pharmaceutical product.

It was an object of the present invention to provide a new form of 4-[(7-chloro-2-methoxybenzo[b][1,5]naphthyridin-10-yl)amino]-2,6-bis(pyrrolidin-1 -ylmethyl)phenol, which shows solid-state properties salts that are beneficial for pharmaceutical development such as in particular a lower weight fraction of inactive mass in the API entity and an improved water uptake behavior.

### DESCRIPTION OF THE INVENTION

The present invention provides novel crystalline forms of 4-[(7-chloro-2-methoxybenzo[b][1,5]naphthyridin-10-yl)amino]-2,6-bis(pyrrolidin-1 -ylmethyl)phenol, which have solid-state properties that are beneficial for pharmaceutical development. In particular the novel crystalline forms of the present invention have little or no inactive mass in the API entity and they show an improved water uptake behavior (compared to the commercially available tetraphosphate salt), which is advantageous in term of pharmaceutical development.

In particular, the present invention provides following novel solid state forms:
Novel crystalline Tosylate salt form - termed Form I;
Novel crystalline Besylate salt form - termed Form II;
Novel crystalline Hemi-Edisylate salt form - termed Form III;
Novel crystalline Napsylate salt form - termed Form IV;
Novel crystalline Napsylate salt form - termed Form V;
Anhydrous free base Form VI; and
Anhydrous free base Form VII.

Unless stated otherwise, the present invention describes solid state forms of pyronaridine and salts thereof, which are chemically pure (chemical purity ≥98% according to NMR analysis).

All forms can be characterized according to standard methods which can be found in e.g. in Rolf Hilfiker, ‘Polymorphism in the Pharmaceutical Industry’, Wiley-VCH. Weinheim 2006 (Chapter 6: X-Ray Diffraction, Chapter 6: Vibrational Spectroscopy, Chapter 3: Thermal Analysis, Chapter 9: Water Vapour Sorption, and references therein) and H.G. Brittain, ‘Polymorphism in Pharmaceutical Solids, Vol. 95, Marcel Dekker Inc., New York 1999 (Chapter 6 and references therein).

As used herein, unless stated otherwise, the X-ray powder diffractogram measurements are taken using monochromatic Cu-Kα₁ radiation wavelength 1.5406 Å. Furthermore, unless stated otherwise, the X-ray powder diffractogram measurements are taken at room temperature.

Solid-state forms of pyronaridine and salts thereof comprise crystal forms or crystalline forms. As used herein, solid-state forms, crystal forms, crystalline forms, polymorphs and polymorphic forms are used interchangeably.

Crystal form may be referred to herein as being characterized by graphical data “substantially as depicted in” or “as depicted in” a Figure. Such graphical data includes for example powder X-ray diffractograms and DSC or TGA. The graphical data potentially provides additional technical information useful to define a particular solid-state form which cannot or not easily be described by reference to numerical values for peak positions and/or relative intensities. The skilled person understands that such graphical representations of data may be subject to small variations, e.g. relative peak intensities and peak positions may vary due to factors such as variations in instrument response and sample concentration and purity. The skilled person would readily be capable of comparing the graphical data shown in the Figures herein with graphical data generated for an unknown crystal form and confirm whether the two sets of graphical data are characterizing the same crystal form or two different crystal forms.

A solid-state form may referred to herein as being characterized by analytical data selected from more different data groupings, such as for example by a X-ray powder diffractogram pattern having a group of specific peaks, or by a X-ray powder diffractogram as shown in a figure, or by “a combination thereof” (or “combinations of these data”). These expressions, e.g., “combination thereof” contemplates that skilled person may characterize a solid state form using any combination of the recited characteristic analytical data. For example, the skilled person may characterize a crystal form using a group, for example, four, five or six characteristic X-ray powder diffractogram peaks, and supplement that characterization with one or more additional features observed in the powder diffractogram, for example an additional peak, characteristic peak shape, peak intensity, or even the absence of a peak at some position in the powder X-ray powder diffractogram pattern. Alternatively, a skilled person may characterize the crystal form using a group of, for example, four, five, six, seven, eight, nine or ten characteristic powder X-ray powder diffractogram peaks, and supplement that characterizing data with one or more additional features observed using another analytical method, for example, using characteristics of the DSC thermogram of the crystal form that is being characterized.

Crystal form (or polymorph) described herein are pure or substantially free of any other crystalline (or polymorphic form). As used herein means that the crystalline form contains 10% or less, of any other known form of the subject compound as measured for example by PXRD.

As used herein, unless stated otherwise, the term “powder” refers to a solid compound in the form of particles or granules, wherein the particles or granules can be poured.

As used herein, unless stated otherwise, the DSC measurements are carried out using a Mettler-Toledo DSC 821 with a heating rate of 5 K/min, using nitrogen purge gas at 50 mL/min.

As used herein, unless stated otherwise, the TGA measurements are carried out using a Mettler-Toledo TGA 851 with a heating rate of 5 K/min, using nitrogen purge gas at 50 mL/min.

As used herein, unless stated otherwise, Water Vapour Sorption isotherm measurements are carried out on a DVS-1 or a DVS-Intrinsic system from SMS.

As used herein and unless stated otherwise, the term “anhydrous” refers to crystalline material which contains not more than 1% (w/w) of either water or organic solvents as measured by TGA. In the context of the present invention, an anhydrous solid state form of a compound refers to a form that does not contain crystal water (or other solvents) in a defined amount within the crystal.

As used herein, unless stated otherwise, the term “solvate” refers to a crystal form that incorporates a solvent in the crystal structure. When the solvent is water, such a form is often referred to as a “hydrate”.

The following abbreviations refer to the abbreviations used above and below: iso-BuOH (iso-butanol), n-BuOH (n-butanol), dec (decomposition), DSC (differential scanning calorimetry), DI (de-ionized), DMSO (dimethyl sulfoxide) EtOH (ethanol), FeSSIF (Fed State Simulated Intestinal Fluid), FaSSIF (Fasted-State Simulated Intestinal Fluid), g (gram), HPLC (high performance liquid chromatography), hr (hour), MHz (Megahertz), MeOH (methanol), min (minute), mL (milliliter), mmol (millimole), mM (millimolar), mp (melting point), MS (mass spectrometry), MW (microwave), NMR (Nuclear Magnetic Resonance), Ph. Eur. (Pharmacopoea Europaea), PTFE (Polytetrafluoroethylene), 2-PrOH (2-propanol), RH (relative humidity), RT (room temperature), TGA (thermal gravimetric analysis), THF (tetrahydrofuran), TMS (trimethylsilyl), UV (ultraviolet), wt% (weight percent), X-ray powder diffractogram (XRPD), Powder X-ray diffraction (PXRD).

In one aspect, the present invention provides a crystalline form of 4-[(7-chloro-2-methoxybenzo[b][1,5]naphthyridin-10-yl)amino]-2,6-bis(pyrrolidin-1-ylmethyl)phenol, designated as Form I. Crystalline Form I is a tosylate salt and can be characterized by following data:
a) a powder X-ray diffraction pattern having one, two, three, four or five peaks at a diffraction angle (2 theta) of 7.1° ± 0.2°, 12.9° ± 0.2°, 15.4° ± 0.2°, 18.2° ± 0.2° and/or 21.2° ± 0.2°;
b) a powder X-ray diffraction pattern having a peak at a diffraction angle 2 theta of 7.1° ± 0.2°, 12.9° ± 0.2°,15.4° ± 0.2°, 18.2° ± 0.2° and/or 21.2° ± 0.2°;and also having one, two, three, four or five additional peaks a diffraction angle 2 theta of 11.9° ± 0.2°, 14.1° ± 0.2°, 14.6° ± 0.2°, 16.7° ± 0.2°and/or 19.3° ± 0.2°;
c) a powder X-ray diffraction pattern according to Table Form I; or
d) a XRPD pattern substantially as depicted in Fig. Form Ia.
and by a combination of these data.

A peak list corresponding to the XRPD of Fig. Form Ia is shown in Table Form I.

TABLE Form I

Powder X-ray peak list of Form I

| No. | °2 θ (Cu-Kα₁ radiation) ± 0.2° |
|---|---|
| 1 | 7.1 |
| 2 | 9.1 |
| 3 | 11.9 |
| 4 | 12.9 |
| 5 | 14.1 |
| 6 | 14.6 |
| 7 | 15.4 |
| 8 | 16.1 |
| 9 | 16.7 |
| 10 | 17.6 |
| 11 | 18.2 |
| 12 | 18.8 |
| 13 | 19.3 |
| 14 | 20.2 |
| 15 | 21.2 |
| 16 | 21.9 |
| 17 | 22.8 |
| 18 | 26.7 |

Tosylate salt Form I can be further characterized by following physical properties:
Tosylate content: 1.05 eq. Tosylate;
Thermal behaviour of Tosylate Form I shows small endothermic events ~135° C. and a melting point at 151° C. DSC and TGA profiles are displayed in Fig. Form Ib and Form Ic. TTGA reveals very small weight loss <1 wt% prior to decomposition.
Water Vapour Sorption behaviour of Form I reveals moderate water uptake levels 2.4 wt% in the relative humidity (rh) range 40-80% rh. Tosylate salt Form I can be classified as hygroscopic acc. to Ph. Eur. criteria based on water uptake difference 40-80% rh (section 5.11.). Water Vapor Sorption isotherm (25° C.) of Form I is displayed in Fig. Form Id.
Dissolution level of Form I in Fasted-State Simulated Intestinal Fluid [FaSSIF, pH 6.5] at 37° C. was determined to be approx. 0.56 mg/mL (after 15 min), approx. 0.55 mg/mL (after 60 min), and approx. 0.54 mg/mL (after 120 min), respectively.

Form I is an anhydrate form which shows a very good crystallinity.

In another aspect, the present invention provides a crystalline form of 4-[(7-chloro-2-methoxybenzo[b][1,5]naphthyridin-10-yl)amino]-2,6-bis(pyrrolidin-1-ylmethyl)phenol, designated as Form II. Crystalline Form II is a besylate salt and can be characterized by following data:
a) a powder X-ray diffraction pattern having one, two, three, four or five peaks at a diffraction angle (2 theta) of 7.8° ± 0.2°, 15.0° ± 0.2°, 17.6° ± 0.2°, 20.7° ± 0.2°and/or 23.3° ± 0.2°;
b) a powder X-ray diffraction pattern having a peak at a diffraction angle 2 theta of 7.8° ± 0.2°, 15.0° ± 0.2°, 17.6° ± 0.2°, 20.7° ± 0.2°and/or 23.3° ± 0.2°and also having one, two, three, four or five additional peaks a diffraction angle 2 theta of 8.7° ± 0.2°, 12.6° ± 0.2°, 16.1° ± 0.2°, 18.3° ± 0.2° and/or 19.1° ± 0.2°;
c) a powder X-ray diffraction pattern according to Table Form II; or
d) a XRPD pattern substantially as depicted in Fig. Form II.
and by a combination of these data.

A peak list corresponding to the XRPD of Fig. Form IIa is shown in Table Form II.

TABLE Form II

Powder X-ray peak list of Form II

| No. | °2θ (Cu-Kα₁ radiation) ± 0.2° |
|---|---|
| 1 | 7.8 |
| 2 | 8.7 |
| 3 | 12.6 |
| 4 | 15 |
| 5 | 16.1 |
| 6 | 17.6 |
| 7 | 18.3 |
| 8 | 19.1 |
| 9 | 20.7 |
| 10 | 21.4 |
| 11 | 23.3 |
| 12 | 24 |
| 13 | 24.7 |
| 14 | 25.5 |
| 15 | 26.1 |
| 16 | 7.8 |

Besylate salt for Form II can be further characterized by following physical properties:
Besylate content: 1.15 eq. Besylate;
Thermal behaviour of Form II shows a small endothermic events at 126° C. and a melting point at 171° C. TGA reveals very small weight loss <1 wt% prior to decomposition. DSC and TGA profiles are displayed in Fig. Form IIb Form IIc.
Water Vapour Sorption behaviour of Form II reveals very small water uptake levels 1.4 wt% in the relative humidity (rh) range 40-90% rh. Besylate salt Form II can be classified as slightly hygroscopic acc. to Ph. Eur. criteria based on water uptake difference 40-80% rh (section 5.11.). Water Vapor Sorption isotherm (25° C.) of Form II is displayed in Fig. Form IId.
Dissolution level of Form II in Fasted-State Simulated Intestinal Fluid [FaSSIF, pH 6.5] at 37° C. was determined to be approx. 0.45 mg/mL (after 15 min), approx. 0.44 mg/mL (after 60 min), and approx. 0.44 mg/mL (after 120 min), respectively.

Form II is an anhydrate form which shows a very good crystallinity.

In another aspect, the present invention provides a crystalline form of 4-[(7-chloro-2-methoxybenzo[b][1,5]naphthyridin-10-yl)amino]-2,6-bis(pyrrolidin-1-ylmethyl)phenol, designated as Form III. Crystalline Form III is a hemi-edisylate and can be characterized by following data:
a) a powder X-ray diffraction pattern having one, two, three, four or five peaks at a diffraction angle (2 theta) of 10.2° ± 0.2°, 13.8° ± 0.2°, 15.1° ± 0.2°, 18.8° ± 0.2° and/or 19.9° ± 0.2°;
b) a powder X-ray diffraction pattern having a peak at a diffraction angle 2 theta of 10.2° ± 0.2°, 13.8° ± 0.2°, 15.1° ± 0.2°, 18.8° ± 0.2° and/or 19.9° ± 0.2° and also having one, two, three, four or five additional peaks a diffraction angle 2 theta of 11.8° ± 0.2°, 17.7° ± 0.2°, 20.6° ± 0.2°, 21.2° ± 0.2° and/or 23.2;
c) a powder X-ray diffraction pattern according to Table Form III; or
d) a XRPD pattern substantially as depicted in Fig. Form III a.
and by a combination of these data.

A peak list corresponding to the XRPD of Fig. Form IIIa is shown in Table Form III.

TABLE Form III

Powder X-ray peak list of Form III

| No. | °2 θ (Cu-Kα₁ radiation) ± 0.2° |
|---|---|
| 1 | 10.2 |
| 2 | 11.8 |
| 3 | 12.5 |
| 4 | 13.8 |
| 5 | 15.1 |
| 6 | 15.5 |
| 7 | 15.9 |
| 8 | 17.7 |
| 9 | 18.8 |
| 10 | 19.2 |
| 11 | 19.9 |
| 12 | 20.3 |
| 13 | 20.6 |
| 14 | 21.2 |
| 15 | 21.7 |
| 16 | 22.5 |
| 17 | 23.2 |
| 18 | 23.7 |

Hemi-edisylate salt Form III can be further characterized by following physical properties:
Edisylate content: 0.5 eq. Edisylate
Thermal behaviour shows overlapped melting / decomposition at 220° C. TGA reveals very small weight loss <1 wt% prior to decomposition. DSC and TGA profiles are displayed in Fig. Form IIIb and Form IIIc.
Water Vapour Sorption behaviour of Form III reveals water uptake levels of 1.5 wt% in the relative humidity (rh) range 40-80% rh. Form IIIcan be classified as slightly hygroscopic acc. to Ph. Eur. criteria based on water uptake difference 40-80% rh (section 5.11.). Water Vapor Sorption isotherm (25° C.) of Form III is in Fig. Form IIId.
Dissolution level of Form IIIin Fasted-State Simulated Intestinal Fluid [FaSSIF, pH 6.5] at 37° C. was determined to be approx. 0.46 mg/mL (after 15 min), approx. 0.48 mg/mL (after 60 min), and approx. 0.47 mg/mL (after 120 min), respectively.

Form III is an anhydrate form which shows a very good crystallinity. Even though Form III is slightly hygroscopic acc. to Ph. Eur. this form shows no tendency to undergo hydrate formation upon exposure to elevated RH levels (up to 90% RH).

In another aspect, the present invention provides a crystalline form of 4-[(7-chloro-2-methoxybenzo[b][1,5]naphthyridin-10-yl)amino]-2,6-bis(pyrrolidin-1-ylmethyl)phenol, designated as Form IV. Crystalline Form IV is a napsylate salt and can be characterized by following data:
a) a powder X-ray diffraction pattern having one, two, three, four or five peaks at a diffraction angle (2 theta) of 6.9° ± 0.2°, 12.6° ± 0.2°, 15.0° ± 0.2°, 15.7° ± 0.2° and/or 22.2° ± 0.2°;
b) a powder X-ray diffraction pattern having a peak at a diffraction angle 2 theta of 6.9° ± 0.2°, 12.6° ± 0.2°, 15.0° ± 0.2°, 15.7° ± 0.2° and/or 22.2° ± 0.2° and also having one, two, three, four or five additional peaks a diffraction angle 2 theta of 9.5° ± 0.2°, 17.2° ± 0.2°, 18.7° ± 0.2°, 19.7° ± 0.2° and/or 20.8° ± 0.2°;
c) a powder X-ray diffraction pattern according to Table Form IV; or
d) a XRPD pattern substantially as depicted in Fig. Form IVa.
and by a combination of these data.

A peak list corresponding to the XRPD of Fig. Form IVa is shown in Table Form IV.

TABLE Form IV

Powder X-ray peak list of Form IV

| No. | °2 θ (Cu-Kα₁ radiation) ± 0.2° |
|---|---|
| 1 | 6.6 |
| 2 | 6.9 |
| 3 | 9.5 |
| 4 | 10.9 |
| 5 | 12.6 |
| 6 | 13.9 |
| 7 | 15 |
| 8 | 15.7 |
| 9 | 16.7 |
| 10 | 17.2 |
| 11 | 17.5 |
| 12 | 18.7 |
| 13 | 19 |
| 14 | 19.7 |
| 15 | 20.2 |
| 16 | 20.8 |
| 17 | 21.6 |
| 18 | 22.2 |

Napsylate salt Form IV can be further characterized by following physical properties:
Napsylate content (determined by NMR):1 eq. Napsylate
Thermal behaviour of Form IV shows melting point at ~169° C. TGA reveals very small weight loss <1 wt% prior to decomposition. DSC and TGA profiles are displayed in Fig. Form IVb and Form IVc.
Water Vapour Sorption behaviour of Form IV reveals water uptake levels of 0.6 wt% in the relative humidity (rh) range 40-80% rh. Form IV can be classified as slightly hygroscopic acc. to Ph. Eur. criteria based on water uptake difference 40-80% rh (section 5.11.). Water Vapor Sorption isotherm (25° C.) of Form IV is displayed in Fig. Form IVd.
Dissolution level of Form IV in Fasted-State Simulated Intestinal Fluid [FaSSIF, pH 6.5] at 37° C. was determined to be approx. 0.17 mg/mL (after 15 min), approx. 0.56 mg/mL (after 60 min), and approx. 0.82 mg/mL (after 120 min), respectively.

Form IV is an anhydrate form which shows a very good crystallinity. Even though Form IV is slightly hygroscopic acc. to Ph. Eur. this form shows no tendency to undergo hydrate formation upon exposure to elevated RH levels (up to 90% RH).

In another aspect, the present invention provides a crystalline form of 4-[(7-chloro-2-methoxybenzo[b][1,5]naphthyridin-10-yl)amino]-2,6-bis(pyrrolidin-1-ylmethyl)phenol, designated as Form V. Crystalline Form V is another napsylate form and can be characterized by following data:
a) a powder X-ray diffraction pattern having one, two, three, four or five peaks at a diffraction angle (2 theta) of 6.9° ± 0.2°, 15.7° ± 0.2°, 17.5° ± 0.2°, 22.2° ± 0.2° and/or 25.4 ± 0.2°;
b) a powder X-ray diffraction pattern having a peak at a diffraction angle 2 theta 6.9° ± 0.2°, 15.7° ± 0.2°, 17.5° ± 0.2°, 22.2° ± 0.2° and/or 25.4 ± 0.2°; and also having one, two or three, additional peaks a diffraction angle 2 theta of 20.8° ± 0.2°, 21.7° ± 0.2° and/or 29.3° ± 0.2°;
c) a powder X-ray diffraction pattern according to Table Form V; or
d) a XRPD pattern substantially as depicted in Fig. Form Va.
and by a combination of these data.

A peak list corresponding to the XRPD of Fig. Form Va is shown in Table Form V.

TABLE Form V

Powder X-ray peak list of Form V

| No. | °2θ (Cu-Kα₁ radiation) ± 0.2° |
|---|---|
| 1 | 6.9 |
| 2 | 15.7 |
| 3 | 17.5 |
| 4 | 20.8 |
| 5 | 21.7 |
| 6 | 22.2 |
| 7 | 25.4 |
| 8 | 29.3 |

Napsylate salt Form V can be further characterized by following physical properties:
Napsylate content (determined by NMR): 1 eq. Napsylate
Thermal behaviour of Form V shows melting point at ~169° C. TGA reveals very small weight loss <1 wt% prior to decomposition. DSC and TGA profiles are displayed in Fig. Form Vb and Form Vc
Water Vapour Sorption behaviour of Form V reveals water uptake levels of 0.6 wt% in the relative humidity (rh) range 40-80% rh. Form V can be classified as slightly hygroscopic acc. to Ph. Eur. criteria based on water uptake difference 40-80% rh (section 5.11.). Water Vapor Sorption isotherm (25° C.) of Form V is displayed in Fig. Form Vd.
Dissolution level of From V in Fasted-State Simulated Intestinal Fluid [FaSSIF, pH 6.5] at 37° C. was determined to be approx. 0.17 mg/mL (after 15 min), approx. 0.56 mg/mL (after 60 min), and approx. 0.82 mg/mL (after 120 min), respectively.

Form V is an anhydrate form which shows a very good crystallinity. Form V is non-hygroscopic acc. to Ph. Eur. and shows no tendency to undergo hydrate formation upon exposure to elevated RH levels (up to 90%RH).

In another aspect, the present invention provides a crystalline form of 4-[(7-chloro-2-methoxybenzo[b][1,5]naphthyridin-10-yl)amino]-2,6-bis(pyrrolidin-1-ylmethyl)phenol, designated as Form VI. Crystalline Form VI is a free base form and can be characterized by following data:
a) a powder X-ray diffraction pattern having one, two, three, four or five peaks at a diffraction angle (2 theta) of 6.7° ± 0.2°, 9.3° ± 0.2°, 16.1° ± 0.2°, 19.4° ± 0.2° and/or 25.0° ±
0.2°;
b) a powder X-ray diffraction pattern having a peak at a diffraction angle 2 theta of 6.7° ± 0.2°, 9.3° ± 0.2°, 16.1° ± 0.2°, 19.4° ± 0.2° and/or 25.0° ± 0.2°; and also having one, two, three, four or five additional peaks a diffraction angle 2 theta of 10.0° ± 0.2°, 11.0° ± 0.2°, 13.4° ± 0.2°, 14.5° ± 0.2° and/or 20.7° ± 0.2°;
c) a powder X-ray diffraction pattern according to Table Form VI; or
d) a XRPD pattern substantially as depicted in Fig. Form VIa.
and by a combination of these data.

A peak list corresponding to the XRPD of Fig. Form VIa is shown in Table Form VI.

TABLE Form VI

Powder X-ray peak list of Form VI

| No. | °2θ (Cu-Kα₁ radiation) ± 0.2° |
|---|---|
| 1 | 6.7 |
| 2 | 9.3 |
| 3 | 10.0 |
| 4 | 11.0 |
| 5 | 12.0 |
| 6 | 13.4 |
| 7 | 14.5 |
| 8 | 15.7 |
| 9 | 16.1 |
| 10 | 17.2 |
| 11 | 17.9 |
| 12 | 18.8 |
| 13 | 19.0 |
| 14 | 19.4 |
| 15 | 20.1 |
| 16 | 20.7 |
| 17 | 24.7 |
| 18 | 25.0 |
| 19 | 27.0 |

Free base Form VI can be further characterized by following physical properties:
Thermal behaviour of Form VI shows small endothermic event ~174° C., followed by exothermic event at ~176° C. and a melting point at 183° C. TGA reveals very small weight loss <1 wt% prior to decomposition. DSC and TGA profiles are displayed in Fig. Form VIb and Form VIc.
Water Vapour Sorption behaviour of Form VI reveals water uptake levels <0.1 wt% in the relative humidity (rh) range 40-80% rh. Form VI can be classified as non-hygroscopic acc. to Ph. Eur. criteria based on water uptake difference 40-80% rh (section 5.11.). Water Vapor Sorption isotherm (25° C.) of free base Form VI is displayed in Fig. Form VId.

Form VI is an anhydrate form which shows a very good crystallinity. Form VI is non-hygroscopic acc. to Ph. Eur. and shows no tendency to undergo hydrate formation upon exposure to elevated RH levels (up to 98% RH).

In another aspect, the present invention provides a crystalline form of 4-[(7-chloro-2-methoxybenzo[b][1,5]naphthyridin-10-yl)amino]-2,6-bis(pyrrolidin-1-ylmethyl)phenol, designated as Form VII.

Crystalline Form VII can be characterized by following data:
a) a powder X-ray diffraction pattern having one, two, three, four or five peaks at a diffraction angle (2 theta) of 6.7° ± 0.2°, 9.9° ± 0.2°, 16.3° ± 0.2°, 19.1° ± 0.2°, and/or 24.3° ± 0.2°;
b) a powder X-ray diffraction pattern having a peak at a diffraction angle 2 theta of 6.7° ± 0.2°, 9.9° ± 0.2°, 16.3° ± 0.2°, 19.1° ± 0.2°, and/or 24.3° ± 0.2°and also having one, two, three or four additional peaks a diffraction angle 2 theta of 17.6° ± 0.2°, 19.8° ± 0.2°, 25.0° ± 0.2° and/or 25.5° ± 0.2°;
c) a powder X-ray diffraction pattern according to Table Form VII; or
d) a XRPD pattern substantially as depicted in Fig. Form VIIa.
and by a combination of these data.

A peak list corresponding to the XRPD of Fig. Form VIIa is shown in Table Form VII.

TABLE Form VII

Powder X-ray peak list of Form VII

| No. | °2θ (Cu-Kα₁ radiation) ± 0.2° |
|---|---|
| 1 | 6.7 |
| 2 | 9.9 |
| 3 | 16.3 |
| 4 | 17.6 |
| 5 | 19.1 |
| 6 | 19.8 |
| 7 | 24.3 |
| 8 | 25.0 |
| 9 | 25.5 |

Free base Form VII can be further characterized by following physical properties:
Thermal behaviour of Form VII shows a melting point at 183° C. TGA reveals very small weight loss <1 wt% prior to decomposition. DSC and TGA profiles are displayed in Fig. Form VIIb and Form VIIc.
Water Vapour Sorption behaviour of Form VII reveals water uptake levels <0.1 wt% in the relative humidity (rh) range 40-80% rh. Form VII can be classified as non-hygroscopic acc. to Ph. Eur. criteria based on water uptake difference 40-80% rh (section 5.11.). Water Vapor Sorption isotherm (25° C.) of Form VII is displayed in Fig. Form VIId.

Form VII is an anhydrate form which shows a very good crystallinity. Form VII is slightly hygroscopic acc. to Ph. Eur. and shows no tendency to undergo hydrate formation upon exposure to elevated RH levels (up to 98% RH).

In general, anhydrous solid state forms are particular advantageous with regard to the process and/or manufacture of solid drug substances, because anhydrates don’t have any risk of phase conversion due to dehydration upon thermal processing.

In a further aspect of the invention a crystalline compound according to the present invention is provided for use as a medicament.

The invention also relates to a crystalline compound according to the present invention for use in the treatment and/or prevention of malaria.

The treatment and/or prevention of malaria as defined herein includes the treatment and/or prevention of infections caused by Plasmodium falciparum, Plasmodium vivax, Plasmodium ovale, Plasmodium malariae and/or Plasmodium knowlesi.

In addition, the invention relates to a pharmaceutical composition comprising a therapeutically effective amount of at least one crystalline compound according to the present invention. In a specific embodiment, the pharmaceutical composition further comprises at least one additional compound selected from the group consisting of physiologically acceptable excipients, auxiliaries, adjuvants, diluents, carriers and/or additional pharmacologically active substances (active ingredients, drugs) other than the crystalline compounds according to the present invention.

The present invention further encompasses a kit comprising a therapeutically effective amount of at least one crystalline compound according to the present invention and/or at least one pharmaceutical composition according to the present invention and a therapeutically effective amount of at least one further pharmacologically active substance (active ingredient, drug) other than the crystalline compounds according to the present invention.

The present invention further encompasses a method for treating and/or prevention malaria, comprising administering to a human in need of such a treatment a therapeutically effective amount of crystalline compound according to the present invention.

Products of the invention may be used in combination with one or more other pharmacologically active substances (ingredients, drugs) in the treatment, prevention, suppression or amelioration of diseases or conditions for which products of the invention or the other substances have utility. Typically the combination of the drugs is safer or more effective than either drug alone, or the combination is safer or more effective than would it be expected based on the additive properties of the individual drugs. Such other drug(s) may be administered, by a route and in an amount commonly used contemporaneously or sequentially with a product of the invention. When a product of the invention is used contemporaneously with one or more other drugs, a combination product containing such other drug(s) and the product of the invention is preferred. However, combination therapy also includes therapies in which the product of the invention and one or more other drugs are administered on different overlapping schedules. It is contemplated that when used in combination with other active ingredients, the product of the present invention or the other active ingredient or both may be used effectively in lower doses than when each is used alone. Accordingly, the pharmaceutical compositions of the present invention (pharmaceutical compositions as described herein) include those that contain one or more other active ingredients, in addition to a product of the invention.

Examples of other pharmacologically active substances (active ingredients, drugs) that may be administered in combination with a product of the invention, and either administered separately or in the same pharmaceutical composition, include, but are not limited to, antimalarial agents such as in particular following compounds: artemisinin or an artemisinin derivative (such as artemether, artesunate or dihydroartemisinin), mefloquine, quinine, cycloguanil, proguanil, metformin, doxycycline, halofantrine, lumefantrine, pyrimethamine, sulfadoxine, piperaquine, atovaquone, 6-Fluoro-2-(4-morpholin-4-ylmethyl-phenyl)-quinoline-4-carboxylic acid (2-pyrrolidin-1-yl-ethyl)-amide (CAS: 1469439-69-7) (or any pharmaceutically acceptable salt of 6-Fluoro-2-(4-morpholin-4-ylmethyl-phenyl)-quinoline-4-carboxylic acid (2-pyrrolidin-1-yl-ethyl)-amide such as in particular a succinate salt), KAF156 (CAS: 1261113-96-5), Tafenoquine (CAS: 106635-80-7), MMV390048 (CAS: 1314883-11-8), DSM265 (CAS: 1282041-94-4), AZ412 (or MMV253, CAS: 1821293-40-6) and/or SAR121.

The pharmaceutical compositions of the present invention (as described herein) may be administered by any means that achieve their intended purpose. For example, administration may be by oral, parenteral, topical, enteral, intravenous, intramuscular, inhalant, nasal, intraarticular, intraspinal, transtracheal, transocular, subcutaneous, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired. Parenteral administration is preferred. Oral administration is especially preferred.

Suitable dosage forms include, but are not limited to capsules, tablets, pellets, dragees, semi-solids, powders, granules, suppositories, ointments, creams, lotions, inhalants, injections, cataplasms, gels, tapes, eye drops, solution, syrups, aerosols, suspension, emulsion, which can be produced according to methods known in the art.

In general, non-chemical routes for the production of pharmaceutical compositions and/or pharmaceutical preparations comprise processing steps on suitable mechanical means known in the art that transfer one or more products of the invention into a dosage form suitable for administration to a patient in need of such a treatment. Usually, the transfer of one or more products of the invention into such a dosage form comprises the addition of one or more compounds, selected from the group consisting of carriers, excipients, auxiliaries and pharmaceutical active ingredients other than the products of the invention. Suitable processing steps include, but are not limited to combining, milling, mixing, granulating, dissolving, dispersing, homogenizing, casting and/or compressing the respective active and non-active ingredients. Mechanical means for performing said processing steps are known in the art, for example from Ullmann’s Encyclopedia of Industrial Chemistry, 5th Edition.

Particularly suitable for oral use are tablets, pills, coated tablets, capsules, powders, granules, syrups, juices or drops, suitable for rectal use are suppositories, suitable for parenteral use are solutions, preferably oil-based or aqueous solutions, furthermore suspensions, emulsions or implants, and suitable for topical use are ointments, creams or powders. The products of the invention may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, dyes, flavours and/or a plurality of further active ingredients, for example one or more vitamins.

Suitable excipients are organic or inorganic substances, which are suitable for enteral (for example oral), parenteral or topical administration and do not react with the products of the invention, for example water, vegetable oils, benzyl alcohols, alkylene glycols, polyethylene glycols, glycerol triacetate, gelatine, carbohydrates, such as lactose, sucrose, mannitol, sorbitol or starch (maize starch, wheat starch, rice starch, potato starch), cellulose preparations and/or calcium phosphates, for example tricalcium phosphate or calcium hydrogen phosphate, magnesium stearate, talc, gelatine, tragacanth, methyl cellulose, hydroxypropylmethylcellulose, sodium carboxymethylcellulose, polyvinyl pyrrolidone and/or vaseline.

If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries include, without limitation, flow-regulating agents and lubricants, for example, silica, talc, stearic acid or salts thereof, such as magnesium stearate or calcium stearate, and/or polyethylene glycol. Dragee cores are provided with suitable coatings, which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices or to provide a dosage form affording the advantage of prolonged action, the tablet, dragee or pill can comprise an inner dosage and an outer dosage component me latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol, solutions of suitable cellulose preparations such as acetylcellulose phthalate, cellulose acetate or hydroxypropylmethyl-cellulose phthalate, are used. Dye stuffs or pigments may be added to the tablets or dragee coatings, for example, for identification or in order to characterize combinations of active compound doses.

Suitable carrier substances are organic or inorganic substances which are suitable for enteral (e.g. oral) or parenteral administration or topical application and do not react with the novel compounds, for example water, vegetable oils, benzyl alcohols, polyethylene glycols, gelatin, carbohydrates such as lactose or starch, magnesium stearate, talc and petroleum jelly. In particular, tablets, coated tablets, capsules, syrups, suspensions, drops or suppositories are used for enteral administration, solutions, preferably oily or aqueous solutions, furthermore suspensions, emulsions or implants, are used for parenteral administration, and ointments, creams or powders are used for topical application. The products of the invention can also be lyophilized and the lyophilizates obtained can be used, for example, for the production of injection preparations.

The preparations indicated can be sterilized and/or can contain excipients such as lubricants, preservatives, stabilizers and/or wetting agents, emulsifiers, salts for affecting the osmotic pressure, buffer substances, colorants, flavourings and/or aromatizers. They can, if desired, also contain one or more further active compounds, e.g. one or more vitamins.

Other pharmaceutical preparations, which can be used orally include push-fit capsules made of gelatine, as well as soft, sealed capsules made of gelatine and a plasticizer such as glycerol or sorbitol. The push-fit capsules can contain the active compounds in the form of granules, which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

The liquid forms in which the novel compositions of the present invention may be incorporated for administration orally include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatine.

The pharmaceutical preparations can be employed as medicaments in human and veterinary medicine. As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Said therapeutic effective amount of one or more of the products of the invention is known to the skilled artisan or can be easily determined by standard methods known in the art.

The products of the invention and the additional pharmacologically active substances are generally administered analogously to commercial preparations. Usually, suitable doses that are therapeutically effective lie in the range between 0.0005 mg and 1000 mg, preferably between 0.005 mg and 500 mg and especially between 0.5 mg and 100 mg per dose unit. The daily dose is preferably between about 0.001 mg/kg and 10 mg/kg of body weight.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

For the purpose of the present invention, all mammalian species are regarded as being comprised. In a preferred embodiment, such mammals are selected from the group consisting of “primate, human, rodent, equine, bovine, canine, feline, domestic animals, cattle, livestock, pets, cow, sheep, pig, goat, horse, pony, donkey, hinny, mule, hare, rabbit, cat, dog, guinea pig, hamster, rat, mouse”. More preferably, such mammals are humans. Animal models are of interest for experimental investigations, providing a model for treatment of human diseases.

The specific dose for the individual patient depends, however, on the multitude of factors, for example on the efficacy of the specific compounds employed, on the age, body weight, general state of health, the sex, the kind of diet, on the time and route of administration, on the excretion rate, the kind of administration and the dosage form to be administered, the pharmaceutical combination and severity of the particular disorder to which the therapy relates. The specific therapeutic effective dose for the individual patient can readily be determined by routine experimentation, for example by the doctor or physician, which advises or attends the therapeutic treatment.

In the case of many disorders, the susceptibility of a particular cell to treatment with the subject compounds may be determined by in vitro testing. Typically a culture of the cell is combined with a subject compound at varying concentrations for a period of time sufficient to allow the active agents to show a relevant reaction, usually between about one hour and one week. For in vitro testing, cultured cells from a biopsy sample may be used.

The present invention further encompasses a process for manufacturing of a crystalline modification according to the present invention.

A specific embodiment includes a process for manufacturing of crystalline Form I comprising following steps:
Providing a dispersion of, wherein the concentration of the reagents (free base and p-toluenesulfonic acid) is preferably in the range of 50 - 100 mg/mL ;
Agitation of the dispersion at ambient temperature (wherein the temperature is preferably between room temperature and 65° C., more preferably between 30° C. and 55° C.);
Multiple (preferably between 4-10) heating/cooling cycles (ambient temperature to a temperature between 0° C. and 10° C. and the other way round) followed by slurrying at a temperature between 0° C. and 10° C. for a couple of hours (preferably between 3 and 30 h); and
Separation of the solid material and subsequent drying of the solid material at ambient temperature (preferably under nitrogen atmosphere).

Another specific embodiment includes a process for manufacturing of crystalline Form II comprising following steps:
Providing a dispersion of, wherein the free base concentration is preferably in the range of 50 - 100 mg/mL;
Agitation of the dispersion at ambient temperature (wherein the temperature is preferably between room temperature and 65° C., more preferably between 30° C. and 55° C.);
Multiple (preferably between 4-10) heating/cooling cycles (ambient temperature to a temperature between 0° C. and 10° C. and the other way round) followed by slurrying at a temperature between 0° C. and 10° C. for a couple of hours (preferably between 3 and 30 h); and
Separation of the solid material and subsequent drying of the solid material at ambient temperature (preferably under nitrogen atmosphere).

Another specific embodiment includes a process for manufacturing of crystalline Form III comprising following steps:
Providing a dispersion of, wherein the free base concentration is preferably in the range of 50 - 100 mg/mL;
Agitation of the dispersion at ambient temperature (wherein the temperature is preferably between room temperature and 65° C., more preferably between 30° C. and 55° C.);
Multiple (preferably between 4-10) heating/cooling cycles (ambient temperature to a temperature between 0° C. and 10° C. and the other way round) followed by slurrying at a temperature between 0° C. and 10° C. for a couple of hours (preferably between 3 and 30 h); and
Separation of the solid material and subsequent drying of the solid material at ambient temperature (preferably under nitrogen atmosphere).

Another specific embodiment includes a process for manufacturing of crystalline Form IV comprising following steps:
Providing a dispersion of, wherein the free base concentration is preferably in the range of 50 - 100 mg/mL;
Agitation of the dispersion at ambient temperature (wherein the temperature is preferably between room temperature and 65° C., more preferably between 30° C. and 55° C.);
Multiple (preferably between 4-10) heating/cooling cycles (ambient temperature to a temperature between 0° C. and 10° C. and the other way round) followed by slurrying at a temperature between 0° C. and 10° C. for a couple of hours (preferably between 3 and 30 h); and
Separation of the solid material and subsequent drying of the solid material at ambient temperature (preferably under nitrogen atmosphere).

Another specific embodiment includes a process for manufacturing of crystalline Form V comprising following steps:
Providing a dispersion of, wherein the free base concentration is preferably in the range of 50 - 100 mg/mL;
Agitation of the dispersion at ambient temperature (wherein the temperature is preferably between room temperature and 65° C., more preferably between 30° C. and 55° C.);
Multiple (preferably between 4-10) heating/cooling cycles (ambient temperature to a temperature between 0° C. and 10° C. and the other way round) followed by slurrying at a temperature between 0° C. and 10° C. for a couple of hours (preferably between 3 and 30 h); and
Separation of the solid material and subsequent drying of the solid material at ambient temperature (preferably under nitrogen atmosphere).

### BRIEF DESCRIPTION OF THE DRAWINGS

FIGS. Form Ia shows a typical Powder X-ray diffractogram of Form I;

FIGS. Form Ib shows a typical DSC scan of Form I;

FIGS. Form Ic shows a typical TGA scan of Form I;

FIGS. Form Id shows a typical Water Vapour Sorption Isotherm of Form I;

FIGS. Form IIa shows a typical Powder X-ray diffractogram of Form II;

FIGS. Form IIb shows a typical DSC scan of Form II;

FIGS. Form IIc shows a typical TGA scan of Form II;

FIGS. Form IId shows a typical Water Vapour Sorption Isotherm of Form II;

FIGS. Form IIIa shows a typical Powder X-ray diffractogram of Form III;

FIGS. Form IIIb shows a typical DSC scan of Form III;

FIGS. Form IIIc shows a typical TGA scan of Form III;

FIGS. Form IIId shows a typical Water Vapour Sorption Isotherm of Form III;

FIGS. Form IVa shows a typical Powder X-ray diffractogram of Form IV;

FIGS. Form IVb shows a typical DSC scan of Form IV;

FIGS. Form IVc shows a typical TGA scan of Form IV;

FIGS. Form IVdshows a typical Water Vapour Sorption Isotherm Form IV;

FIGS. Form Va shows a typical Powder X-ray diffractogram of Form V;

FIGS. Form Vb shows a typical DSC scan of Form V;

FIGS. Form Vc shows a typical TGA scan of Form V;

FIGS. Form Vd shows a typical Water Vapour Sorption Isotherm Form V;

FIGS. Form VIa shows a typical Powder X-ray diffractogram of Form VI;

FIGS. Form VIb shows a typical DSC scan of Form VI;

FIGS. Form Vic shows a typical TGA scan of Form VI;

FIGS. Form VId shows a typical Water Vapour Sorption Isotherm Form VI;

FIGS. Form VIIa shows a typical Powder X-ray diffractogram of Form VII;

FIGS. Form VIIb shows a typical DSC scan of Form VII;

FIGS. Form VIIc shows a typical TGA scan of 3

FIGS. Form VIId shows a typical Water Vapour Sorption Isotherm Form VII;

FIG. tetraphosphateashows a typical Powder X-ray diffractogram of the tetraphosphate (prior art form);

FIG. tetraphosphateb shows a typical DSC scan of the tetraphosphate (prior art form);

FIG. tetraphosphatec shows a typical TGA scan of tetraphosphate (prior art form); and

FIG. tetraphosphated shows a typical Water Vapour Sorption Isotherm tetraphosphate (prior art form).

The invention is explained in more detail by means of the following examples without, however, being restricted thereto.

### Examples

### Example 1: Prior Art Form - Characterisation of Commercially Available Pyronaridine (Tetraphosphate Form)

NMR data for tetraphosphate form:

¹H NMR (500 MHz, DMSO-d₆) δ 8.23 (d, J = 9.2 Hz, 1H), 7.97 (d, J = 2.2 Hz, 1H), 7.87 (d, J = 9.3 Hz, 1H), 7.32 (d, J = 9.2 Hz, 1H), 7.26 (dd, J = 9.3, 2.2 Hz, 1H), 7.09 (d, J = 2.5 Hz, 2H), 3.97 (s, 4H), 3.88 (s, 3H), 2.84 (s, 9H), 2.09 (s, 1H), 1.83 (d, J = 3.8 Hz, 6H).

A Powder X-Ray Diffraction pattern of the commercially available tetraphosphate form has been obtained by standard techniques as described in the European Pharmacopeia 6^{th} Edition chapter 2.9.33, and is characterised by the X-ray powder diffractogram shown in FIG. tetraphosphate (monochromatic Cu-Kα₁ radiation, λ = 1.5406 Å, Stoe StadiP 611 KL transmission diffractometer).

A peak list corresponding to the X-ray powder diffractogram shown in FIG. tetraphosphate is shown below:

| No. | °2θ (Cu-K α₁radiation) ± 0.2° |
|---|---|
| 1 | 5.6 |
| 2 | 6.4 |
| 3 | 7.8 |
| 4 | 8.9 |
| 5 | 9.9 |
| 6 | 11.5 |
| 7 | 12.6 |
| 8 | 14.2 |
| 9 | 14.7 |
| 10 | 16.1 |
| 11 | 17.1 |
| 12 | 18 |
| 13 | 19.3 |
| 14 | 20.3 |
| 15 | 20.9 |
| 16 | 21.3 |
| 17 | 22.2 |
| 18 | 22.7 |

Tetraphosphate form is characterised by the following physical properties:
Tetraphosphate form exhibits a melting /decomposition point >220° C. TGA scans revealed weight loss step of approx. 2.1 wt% up to 90° C. DSC and TGA profiles are in FIG. tetraphosphaseb and tetraphosphatec.
Water Vapour Sorption behaviour of Tetraphosphate form reveals strong water uptake levels of >4.9 wt% in the relative humidity (rh) range 40-80% rh. Tetraphosphate form can be classified as hygroscopic acc. to Ph. Eur. criteria (section 5.11.), based on water uptake difference between 40% rh and 80% rh. Water Vapor Sorption isotherm (25° C.) of Tetraphosphate form is shown in FIG. tetraphosphated.
Dissolution level of Tetraphosphate form in Fasted-State Simulated Intestinal Fluid [FaSSIF, pH 6.5] at 37° C. was determined to be approx. 0.50 mg/mL (after 15 min), approx. 0.50 mg/mL (after 60 min), and approx. 0.49 mg/mL (after 120 min), respectively.

### Example 2: Preparation Processes for Novel Salt Form I

Approx. 41 mg of 4-[(7-chloro-2-methoxybenzo[b][1,5]naphthyridin-10-yl)amino]-2,6-bis(pyrrolidin-1-ylmethyl)phenol free base and 16 mg of p-Toluenesulfonic acid were dispersed in 4 mL of EtOAc under agitation at 50° C. Then we started multiple cooling-heating cycles (50-4° C. in ~10 hours, 4-50° C. in 0.5 hours) 4-5 cooling/heating cycles and subsequent final slurrying at 4° C. for 24 h. Resulting solid-state residue was separated from supernatant liquid by centrifugation, and dried under nitrogen purge gas at 80° C. for 48 hours to give a powder sample.

¹H NMR (500 MHz, DMSO-d₆) δ 9.07 (s, 1H), 8.24 (d, J = 9.2 Hz, 1H), 7.99 (d, J = 2.2 Hz, 1H), 7.85 (d, J = 9.3 Hz, 1H), 7.48 (d, J = 8.1 Hz, 2H), 7.39 -7.24 (m, 2H), 7.18 - 6.99 (m, 4H), 4.06 (s, 4H), 3.89 (s, 3H), 2.92 (s, 8H), 2.29 (s, 3H), 1.86 (d, J = 3.5 Hz, 6H).

### Example 3: Preparation Processes for Novel Salt Form II

Approx. 40 mg of 4-[(7-chloro-2-methoxybenzo[b][1,5]naphthyridin-10-yl)amino]-2,6-bis(pyrrolidin-1-ylmethyl)phenol free base and 13.2 mg of Benzenesulfonic acid were dispersed in 4 mL of EtOAc under agitation at 50° C. Then we started multiple cooling-heating cycles (50-4° C. in ~10 hours, 4-50° C. in 0.5 hours) 4-5 cooling/heating cycles and subsequent final slurrying at 4° C. for 24 h. Resulting solid-state residue was separated from supernatant liquid by centrifugation, and dried under nitrogen purge gas at 80° C. for 48 hours to give a powder sample.

¹H NMR (500 MHz, DMSO-d₆) δ 9.10 (s, 1H), 8.24 (d, J = 9.2 Hz, 1H), 7.99 (d, J = 2.3 Hz, 1H), 7.86 (d, J = 9.4 Hz, 1H), 7.67 - 7.51 (m, 2H), 7.31 (tdd, J = 6.7, 4.6, 1.5 Hz, 4H), 7.12 (s, 2H), 4.08 (s, 4H), 3.88 (s, 3H), 2.94 (s, 8H), 2.09 (s, 0H), 1.87 (s, 3H).

### Example 4: Preparation Processes for Novel Salt Form III

Approx. 42 mg of 4-[(7-chloro-2-methoxybenzo[b][1,5]naphthyridin-10-yl)amino]-2,6-bis(pyrrolidin-1-ylmethyl)phenol free base and 15.5 mg of ethanedisulfonic acid were dispersed in 4 mL of EtOAc under agitation at 50° C. Then we started multiple cooling-heating cycles (50-4° C. in ~10 hours, 4-50° C. in 0.5 hours) 4-5 cooling/heating cycles and subsequent final slurrying at 4° C. for 24 h. Resulting solid-state residue was separated from supernatant liquid by centrifugation, and dried under nitrogen purge gas at 80° C. for 48 hours to give a powder sample.

¹H NMR (500 MHz, DMSO-d₆) δ 9.09 (s, 1H), 8.24 (d, J = 9.2 Hz, 1H), 7.99 (d, J = 2.3 Hz, 1H), 7.86 (d, J = 9.3 Hz, 1H), 7.40 - 7.23 (m, 2H), 7.11 (s, 2H), 4.07 (s, 4H), 3.89 (s, 3H), 2.94 (s, 8H), 2.63 (s, 2H), 1.87 (t, J = 3.6 Hz, 7H).

### Example 5: Preparation Processes for Novel Salt Form IV

Approx. 39 mg of 4-[(7-chloro-2-methoxybenzo[b][1,5]naphthyridin-10-yl)amino]-2,6-bis(pyrrolidin-1-ylmethyl)phenol free base and 16 mg of Naphtalene-2-sulfonic acid were dispersed in 4 mL of THF under agitation at 50° C. Then we started multiple cooling-heating cycles (50-4° C. in ~10 hours, 4-50° C. in 0.5 hours) 4-5 cooling/heating cycles and subsequent final slurrying at 4° C. for 24 h followed by an antisolvent vapor diffusion overnight. Resulting solid-state residue was separated from supernatant liquid by centrifugation and dried under nitrogen purge gas at 80° C. for 48 hours to give a powder sample.

¹H NMR (500 MHz, DMSO-d₆) δ 8.24 (d, J = 9.2 Hz, 0H), 8.14 (d, J = 1.6 Hz, 0H), 8.02 - 7.94 (m, 0H), 7.93 - 7.80 (m, 0H), 7.72 (dd, J = 8.5, 1.7 Hz, 0H), 7.52 (dd, J = 6.2, 3.2 Hz, 0H), 7.40 - 7.24 (m, 0H), 7.10 (s, 0H), 4.14 - 3.99 (m, 1H), 3.89 (s, 0H), 2.93 (s, 1H), 1.86 (d, J = 4.1 Hz, 1H).

### Example 6: Preparation Processes for Novel Salt Form V

Approx. 39 mg of 4-[(7-chloro-2-methoxybenzo[b][1,5]naphthyridin-10-yl)amino]-2,6-bis(pyrrolidin-1-ylmethyl)phenol free base and 16 mg of Naphtalene-2-sulfonic acid were dispersed in 4 mL of THF under agitation at 50° C. Then we started multiple cooling-heating cycles (50-4° C. in ~10 hours, 4-50° C. in 0.5 hours) 4-5 cooling/heating cycles and subsequent final slurrying at 4° C. for 24 h followed by an antisolvent vapor diffusion overnight. Resulting solid-state residue was separated from supernatant liquid by centrifugation and dried under nitrogen purge gas at 80° C. for 48 hours to give a powder sample.

¹H NMR (500 MHz, DMSO-d₆) δ 8.25 (d, J = 9.2 Hz, 0H), 8.14 (d, J = 1.6 Hz, 0H), 8.03 - 7.94 (m, 0H), 7.93 - 7.77 (m, 0H), 7.72 (dd, J = 8.5, 1.7 Hz, 0H), 7.52 (dd, J = 6.2, 3.2 Hz, 0H), 7.41 - 7.24 (m, 0H), 4.09 (s, 0H), 3.88 (s, 0H), 2.94 (s, 1H), 1.87 (s, 1H).

### Example 7: Non-sink Dissolution Assessment of Salt Forms

Results from miniaturized non-sink dissolution studies for selected forms are summarised below.

Approx. 10-20 mg of solid sample were weighed into glass vials. 7 ml of respective FaSSIF (pH 6.5) medium (prewarmed to 37° C.) were added and the suspension was shaken at 450 rpm at 37° C. After 5 min, 15 min, 60 min, and 120 min, 1 ml suspension was withdrawn and filtered through a 0.2 µm syringe filter. Clear filtrate was analysed by HPLC after suitable dilution to measure the amount of API dissolved. To get non-sink conditions 1 ml pre warmed FaSSiF solution was added after sample was withdrawn.

The clear solution was collected in HPLC vials, the filtrate further diluted if necessary and finally HPLC analysis was carried out.

HPLC method for ph-dependent solubility & miniaturised non-sink dissolution:
Column: Chromolith RP-18e 100 - 3 mm
Solvent A: water/formic acid (999:1; v/v)
Solvent B: acetonitrile/formic acid (999:1; v/v)
Injection volume: 5 µL
Column temperature: 37° C.
Wavelength detector: 218 nm

HPLC-Gradient

| Time (minutes) | Eluent A (%) | Eluent B (%) | Flow (mUmin) |
|---|---|---|---|
| 0.0 | 90 | 10 | 1.70 |
| 0.3 | 90 | 10 | 1.70 |
| 2.0 | 10 | 90 | 1.70 |
| 2.75 | 10 | 90 | 1.70 |
| 2.76 | 90 | 10 | 2.50 |
| 4.00 | 90 | 10 | 2.50 |

| Time | Dissolution levels in FaSSIF pH 6.5 (µg/mL) | |
|---|---|---|
| | Pyronaridine (tetraphosphate salt, prior art form) | Tosylate salt Form I |
| 5 min | 501.4 | 547.9 |
| 15 min | 501.9 | 557.9 |
| 60 min | 501.3 | 552.8 |
| 120 min | 491.3 | 537.7 |

| Time | Dissolution levels in FaSSIF pH 6.5 (µg/mL) | | |
|---|---|---|---|
| | Besylate salt Form II | Edisylate salt Form III | Napsylate salt Form IV |
| 5 min | 432.8 | 406.7 | 116.6 |
| 15 min | 447.0 | 462.7 | 209.0 |
| 60 min | 440.1 | 483.3 | 488.8 |
| 120 min | 444.5 | 469.7 | 630.0 |

| Time | Dissolution levels in FaSSIF pH 6.5 (µg/mL) |
|---|---|
| | Napsylate salt Form V |
| 5 min | 95.2 |
| 15 min | 171.1 |
| 60 min | 564.1 |
| 120 min | 817.6 |

### Example 8: Preparation Processes for Novel Anhydrous Form VI of Free Base

Approx. 20 mg of 4-[(7-chloro-2-methoxybenzo[b][1,5]naphthyridin-10-yl)amino]-2,6-bis(pyrrolidin-1-ylmethyl)phenol free base (material obtained from salt hydrolysis of tetraphosphate salt [pyronaridine tetraphosphate {0.1 g} was dissolved in water {1.0 mL} and passed through a SCX column {500 mg prepacked tosic acid resin}. The column was eluted with water {10 mL} followed by methanol {10 mL} to remove the acidic impurities. Finally, the free base was obtained by treating the column with methanolic ammonia {10 mL} and the eluents were concentrated to obtain the free base) are prepared in a DSC AI pan (100 µL), and heated to 140° C. under a nitrogen atmosphere (50 mL/min) using a linear heating rate of 5 K/min in a DSC instrument. After reaching 140° C., the sample is removed from the DSC cell, and kept at ambient conditions.

### Example 9: Preparation Processes for Novel Anhydrous Form VII of Free Base

Approx. 20 mg of 4-[(7-chloro-2-methoxybenzo[b][1,5]naphthyridin-10-yl)amino]-2,6-bis(pyrrolidin-1-ylmethyl)phenol free base (material obtained from salt hydrolysis of tetraphosphate salt [pyronaridine tetraphosphate {0.1 g} was dissolved in water {1.0 mL} and passed through a SCX column {500 mg prepacked tosic acid resin}. The column was eluted with water {10 mL} followed by methanol {10 mL} to remove the acidic impurities. Finally, the free base was obtained by treating the column with methanolic ammonia {10 mL} and the eluents were concentrated to obtain the free base) are prepared in a DSC AI pan (100 µL), and heated to approx. 175° C. under a nitrogen atmosphere (50 mL/min) using a linear heating rate of 5 K/min in a DSC instrument. After reaching 175° C., the sample is removed from the DSC cell, and kept at ambient conditions.

### Example 10: Comparison of Solubility Data for Tetraphosphate Salt and Free Base Form VI and Form VII

Results from thermodynamic solubility studies for selected forms are summarised below.

Approx. 5 mg of solid sample were weighed into a 4 ml glass vial. 1 ml of respective aqueous buffer was added and the suspension was shaken for 24 h at 450 rpm at 37° C. After 1 h, 6 h and after 24 h the vials were checked for presence of undissolved compound and the pH was measured. If necessary, the pH was adjusted. After 24 h the solid liquid separation was carried out using 1 ml syringe and 0.2 µm syringe filter. Clear filtrate was analysed by HPLC after suitable dilution to measure the amount of API dissolved.

HPLC method for ph-dependent solubility and miniaturised non-sink dissolution:
Column: Chromolith RP-18e 100 - 3 mm
Solvent A: water/formic acid (999:1; v/v)
Solvent B: acetonitrile/formic acid (999:1; v/v)
Injection volume: 5 µL
Column temperature: 37° C.
Wavelength detector: 218 nm

HPLC-Gradient

| Time (minutes) | Eluent A (%) | Eluent B (%) | Flow (mL/min) |
|---|---|---|---|
| 0.0 | 90 | 10 | 1.70 |
| 0.3 | 90 | 10 | 1.70 |
| 2.0 | 10 | 90 | 1.70 |
| 2.75 | 10 | 90 | 1.70 |
| 2.76 | 90 | 10 | 2.50 |
| 4.00 | 90 | 10 | 2.50 |

1. A crystalline form of 4-[(7-chloro-2-methoxybenzo[b][1,5]naphthyridin-10-yl)amino]-2,6-bis(pyrrolidin-1-ylmethyl)phenol, which represents a sulfonate salt or a free base form.

2. The crystalline form of 4-[(7-chloro-2-methoxybenzo[b][1,5]naphthyridin-10-yl)amino]-2,6-bis(pyrrolidin-1-ylmethyl)phenol according to claim 1, wherein the crystalline form is designated as a Form I, wherein the Form I has a powder X-ray diffraction pattern having one, two, three, four, or five peaks at a diffraction angle (2 theta) of 7.1 ° ± 0.2°. 12.9° ± 0.2°,15.4° ± 0.2°, 18.2° ± ().2°, and/or 21.2° ± 0.2°.

3. The crystalline form of 4-[(7-chloro-2-methoxybenzo[b][1,5]naphthyridin-10-yl)amino]-2,6-bis(pyrrolidin-1-ylmethyl)pheno( according to claim 1, wherein the crystalline form is designated as a Form II, wherein the Form II has a powder X-ray diffraction pattern having one, two, three, four, or five peaks at a diffraction angle (2 theta) of 7.8° ± 0.2°. 15.0° ± 0.2°, 17.6° ± 0.2°, 20.7° ± 0.2°, and/or 23.3° ± 0.2°.

4. The crystalline form of 4-(7-chloro-2-methoaybenro[b][1,5]naphthyridin-10-yl)amino]-2,6-bis(prrolidin-1-ylmethyl)phenol according to claim 1, wherein the crystalline form is designated as a Form III, wherein the Form III has a powder X-ray diffraction pattern having one, two, three, four, or five peaks at a diffraction angle (2 theta) of 10.2° ± 0.2°, 13.8° ± 0.2°, 15.1 ° ± 0.2°, 18.8° ± 0.2°, and/or 19.9° ± 0.2°.

5. The crystalline form of 4-[(7-chloro-2-methoxybenzo[b][1,5]naphlhyridin-10yl)amino]-2,6-bis(pyrrolidin-1-ylmethyl)phenol according to claim 1, wherein the crystalline form is designated as a Form IV, wherein the Form IV has a powder X-ray diffraction pattern having one, two, three, four, or five peaks at a diffraction angle (2 theta) of 6.9° ± 0.2°, 12.6° ± 0.2°, 15.0° ± 0.2°, 15.7° ± 0.2°, and/or 22.2° ± 0.2°.

6. The crystalline form of 4-[(7-chloro-2-methoxybenzo[b][1,5]naphthyridin-10-yl)amino]-2,6-bis(pyrrolidin-1-ylmethyl)phenol according to claim 1, wherein the crystalline form is designated as a Form V, wherein the Form V has a powder X-ray diffraction pattern having one, two, three, four, or five peaks at a diffraction angle (2 theta) of6.9° ± 0.2°, 15.7° ± 0.2°, 17.5° ± 0.2°. 22.2° ± 0.2°, and/or 25.4 ± 0.2°.

7. The crystalline form of 4-[(7-chloro-2-methoxybenzo[b][1,5](naphthyridin-10-yl)amino]-2,6-bis(pyrrolidin-1-ylmethyl)phenol according to claim 1, wherein the crystalline form is designated as a Form VI, wherein the Form VI has a powder X-ray diffraction pattern having one, two, three, four, or five peaks at a diffraction angle (2 theta) 6.7° ± 0.2°, 9.3° ± 0.2°, 16.1° ± 0.2°, 19.4° ± 0.2°, and/or 25.0° ± 0.2°.

8. The crystalline form of 4-[(7-chloro-2-methoxybenzo[b][1,5]naphthyridin-10-yl]amino-2,6-bis(pyrrolidin-1-ylmethyl)phenol according to claim 1, wherein the crystalline form is designated as a Form VII, wherein the Form VII has a powder X-ray diffraction pattern having one, two, three, four, or five peaks at a diffraction angle (2 theta) of 6.7° ± 0.2°, 9.9° ± 0.2°, 16.3° ± 0.2°, 19.1° ± 0.2°, and/or 24.3° ± 0.2.

9. A medicament, comprising the crystalline form according to claim 1.

10. A method of treatment of a parasitic infection, the method comprising:
administering the crystalline form according to claim 1 to a patient in need thereof.

11. A pharmaceutical composition, comprising:
a therapeutically effective amount of at least one crystalline form according to claim 1.

12. The pharmaceutical composition according to claim 11, further comprising.
at least one additional compound selected from the group consisting of a physiologically acceptable excipient, auxiliary, adjuvant, diluent, and carrier; and a pharmaceutically active substance other than the at least.

13. A kit, comprising:
a therapeutically effective amount of at least one crystalline form according to claim 1, and/or at least one pharmaceutical composition comprising a therapeutically effective amount of the at least one crystalline form and
a therapeutically effective amount of at least one further pharmacologically active.

14. A method for treating comprising:
administering to a human in need of such a treatment a therapeutically effective amount of the crystalline form according to claim 1.

15. A process, comprising:
manufacturing the crystalline form according to claim 1.

16. The method according to claim 10, wherein the parasitic infection is malaria or a parasitic infection caused by a plasmodium species.

17. The pharmaceutical composition according to claim 11, further comprising:
a therapeutically effective amount of a second pharmacologically active substance.

18. The pharmaceutical composition according to claim 17, wherein the second pharmacologically active substance is an antimalarial.

19. The kit according to claim 13, wherein the at least one further pharmacologically active substance comprises an antimalarial agent.
